(19) United States
(12) Patent Application Publication
(10) Pub. No.: US 2026/0069265 A1
(43) Pub. Date: Mar. 12, 2026

(54) PUNCTURE LOCKING DEVICE AND SUTURE APPARATUS

(71) Applicant: Halocinch Medical Technology (Shenzhen) Co., Ltd., Shenzhen (CN)

(72) Inventors: Qiao HUANG, Shenzhen (CN); Qizong XIE, Shenzhen (CN); Jiayang LI, Shenzhen (CN)

(21) Appl. No.: 19/386,522

(22) Filed: Nov. 12, 2025

Related U.S. Application Data
(63) Continuation-in-part of application No. PCT/CN2024/095716, filed on May 28, 2025.

(30) Foreign Application Priority Data
May 31, 2023 (CN) 202310628025.5

(51) Int. Cl. A61B 17/04; A61B 17/00; A61B 90/00

(52) U.S. Cl. CPC A61B 17/0469; A61B 90/03; A61B 2017/00862; A61B 2017/047; A61B 2090/034

(57) ABSTRACT

Disclosed in the present application are a puncture locking device and a suture apparatus. The suture apparatus includes a catheter, a handheld operation portion, and the puncture locking device. The puncture locking device includes a suture head, and a suture assembly and a locking structure are arranged inside the suture head. The suture assembly includes a suture needle and a push-pull guiding member, and the push-pull guiding member moves in an axial direction of the catheter so as to drive the suture needle to move. The locking structure includes an elastic locking assembly and an unlocking member, the elastic locking assembly includes a locking portion and an elastic portion. The handheld operation portion can control the movement of the push-pull guiding member and the unlocking member. By means of the locking structure, the suture needle is locked, such that pre-triggering of the suture needle caused by misoperation is avoided.

The present application is a continuation in part of International Application No. PCT/CN2024/095716 filed on May 28, 2025, which claims the priority of the Chinese patent application No. 202310628025.5, filed on May 31, 2023, and entitled “puncture locking device and suture apparatus”, which are incorporated herein by reference in their entireties.

### FIELD

The subject matter relates to the field of medical devices, and more particularly, to a puncture locking device and a suture apparatus.

### BACKGROUND

Structural heart disease refers to a condition where pathological phenomena occur in the heart. Currently, transcatheter interventional suturing for repairing diseased tissue is the approach closest to surgical repair for the structural heart disease. The transcatheter interventional suturing can maximally preserve the integrity of the cardiac anatomy, with minimal trauma and the least implants, making it the most promising solution for repairing the structural heart disease.

The transcatheter interventional suturing involves introducing a suture needle into a patient's body through a catheter to suture the tissue. However, interventional suture devices in the related arts may not be able to lock the suture needle. Thus, the suture needle may easily be prematurely triggered in cardiovascular tissue due to misoperation, resulting in damages to the heart tissue which cause harm to the patient.

### SUMMARY

The present application provides a puncture locking device including a suture head. A suture assembly and a locking structure are arranged inside the suture head. The suture assembly includes a suture needle and a push-pull guiding member operatively coupled to the suture needle. The locking structure includes an elastic locking assembly and an unlocking member. The elastic locking assembly includes a locking portion for restricting movement of the push-pull guiding member and an elastic portion for providing a locking force to the locking portion. In a locked state, the elastic portion applies a force to the locking portion such that the locking portion tilts and abuts against the push-pull guiding member, thereby restricting movement of the push-pull guiding member in a first direction. The first direction is the direction in which the push-pull guiding member moves to cause the suture needle to penetrate out of the suture head. In an unlocked state, the unlocking member moves within the suture head to counterbalance the force applied by the elastic portion to the locking portion, thereby unlocking the push-pull guiding member.

The present application also provides a suture apparatus. The suture apparatus includes a catheter, a handheld operation portion disposed at a proximal end of the catheter, and the puncture locking device described above. The suture head is disposed at a distal end of the catheter. The push-pull guiding member extends through the catheter and is capable of reciprocating movement along an axial direction of the catheter. The unlocking member extends through the catheter and is capable of moving along the axial direction of the catheter. Both the push-pull guiding member and the unlocking member are connected to the handheld operation portion. The handheld operation portion is configured to operably control movement of the push-pull guiding member and the unlocking member.

### BRIEF DESCRIPTION OF THE DRAWINGS

In order to clearly illustrate the present technology, a brief description of the drawings used in various embodiments or exemplarily implementations is given as follows. Obviously, the drawings are only some embodiments of the present disclosure. For those of ordinary skill in the art, other drawings can be obtained based on these drawings without creative work.

FIG. 1 is a diagram of a suture apparatus according to an embodiment of the present disclosure.

FIG. 2 is a diagram of a suture head of the suture apparatus according to an embodiment of the present disclosure.

FIG. 3 is an exploded view of a puncture locking device according to an embodiment of the present disclosure.

FIG. 4 is a cross-sectional view of the suture head according to an embodiment of the present disclosure.

FIG. 5 is a cross-sectional view showing a suture needle penetrating out of the suture head according to an embodiment of the present disclosure.

FIG. 6 is a diagram showing a locking block disposed on one side of a push-pull guiding member and in a locked state with the push-pull guiding member according to an embodiment of the present disclosure.

FIG. 7 is a diagram showing the locking block pivotably disposed within the suture head and in a locked state with the push-pull guiding member according to an embodiment of the present disclosure.

FIG. 8 is a diagram showing the locking block in a locked state with the push-pull guiding member when the push-pull guiding member extends through the locking block according to an embodiment of the disclosure.

FIG. 9 is a diagram showing the locking block in an unlocked state with the push-pull guiding member when the push-pull guiding member extends through the locking block according to an embodiment of the present disclosure.

FIG. 10 is a diagram of an unlocked state when an elastic portion employs an elastic sheet according to an embodiment of the present disclosure.

FIG. 11 is a diagram of a locked state when the elastic portion employs the elastic sheet according to an embodiment of the present disclosure.

FIG. 12 is a diagram of a locked state when an unlocking member is in threaded engagement with the suture head according to an embodiment of the present disclosure.

FIG. 13 is a diagram of an unlocked state when the unlocking member is in threaded engagement with the suture head according to an embodiment of the present disclosure.

FIG. 14 is a diagram of a locked state when the unlocking member employs a pull wire according to an embodiment of the present disclosure.

FIG. 15 is a diagram of an unlocked state when the unlocking member employs the pull wire according to an embodiment of the present disclosure.

FIG. 16 is a diagram of an unlocked state when a locking portion is a locking hole according to an embodiment of the present disclosure.

FIG. 17 is a diagram of a locked state when the locking portion is the locking hole according to an embodiment of the present disclosure.

FIG. 18 is a diagram showing engagement between the push-pull guiding member and an elastic main body when the locking portion is the locking hole according to an embodiment of the present disclosure.

FIG. 19 is a diagram showing engagement between the locking hole and the push-pull guiding member in a locked state according to an embodiment of the present disclosure.

FIG. 20 is a diagram of the elastic main body in an unlocked state when the locking portion is the locking hole according to an embodiment of the present disclosure.

FIG. 21 is a diagram of the elastic main body in a locked state when the locking portion is the locking hole according to an embodiment of the present disclosure.

FIG. 22 is a diagram of the push-pull guiding member provided with an anti-slip structure according to an embodiment of the present disclosure.

FIG. 23 is a diagram of a handheld operation portion according to an embodiment of the present disclosure.

FIG. 24 is a diagram showing the suture head being intervened into a human heart via a catheter according to an embodiment of the present disclosure.

### DETAILED DESCRIPTION

Implementations of the present disclosure will now be described, by way of embodiments only, with reference to the drawings to make the purpose, technical solutions, and advantages of the present application clear. It should be understood that the embodiments are illustrative only but not considered as limiting the present disclosure.

It should be noted that when a component is referred to as being or “fixed on” or “arranged on” another component, the component can be directly or indirectly on another component. When a component is considered to be “connected to” another component, the component can be directly or indirectly connected to another component. Orientations or positional relationships indicated by terms “on”, “under”, “left”, “right”, are relative to the orientations or positional relationships shown in the attached drawings, are merely for convenience of description, and do not indicate or imply that the referred apparatus or element must have a specific orientation or be constructed and operated in a specific orientation. Therefore, they should not be construed as limiting the present application. Those of ordinary skill in the art can understand the specific meanings of the above terms according to specific situations. The terms “first” and “second” are used for description purposes only and should not be understood as indicating or implying their relative importance or implying the quantity of the indicated technical features. The meaning of “a plurality” is two or more, unless explicitly specified otherwise.

In the technical field of interventional medical devices, an orientation close to an operator is generally defined as a proximal end, and an orientation away from the operator is defined as a distal end. The direction of the central axis of a cylindrical body, or a tubular body, is defined as an axial direction. A radial direction refers to the direction passing through the central axis in a radial plane, for example, the direction along a diameter or radius, or the direction perpendicular to the central axis.

Implementations of the disclosure will now be described, byway of embodiments only, with reference to the drawings.

Currently, transcatheter interventional suturing is considered the most promising solution for repairing structural heart disease. However, interventional suture devices in the related arts may not be able to lock the suture needle. The suture needle may be prematurely triggered due to misoperation within the cardiovascular tissue, resulting in damages to the cardiac tissue which cause harm to the patient.

The present application provides a puncture locking device. By arranging a locking structure in a suture head for locking the push-pull guiding member, the suture needle may be locked, thereby reducing the risk of the suture needle penetrating out of the suture head. Furthermore, the locking structure is disposed within the suture head and close to the suture needle, such that the locking structure can lock the push-pull guiding member at the distal end, and make the locking effect more direct and stable. Additionally, the locking structure is compact in structure and does not increase the overall size of the instrument.

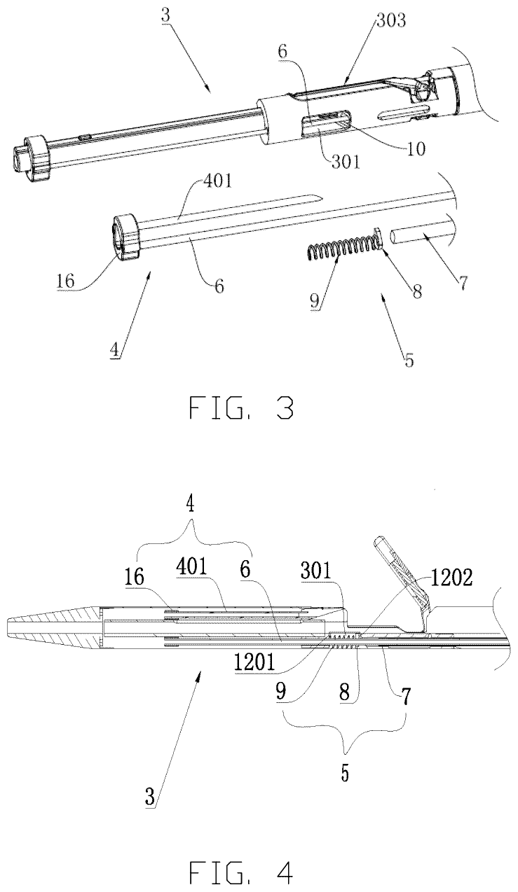

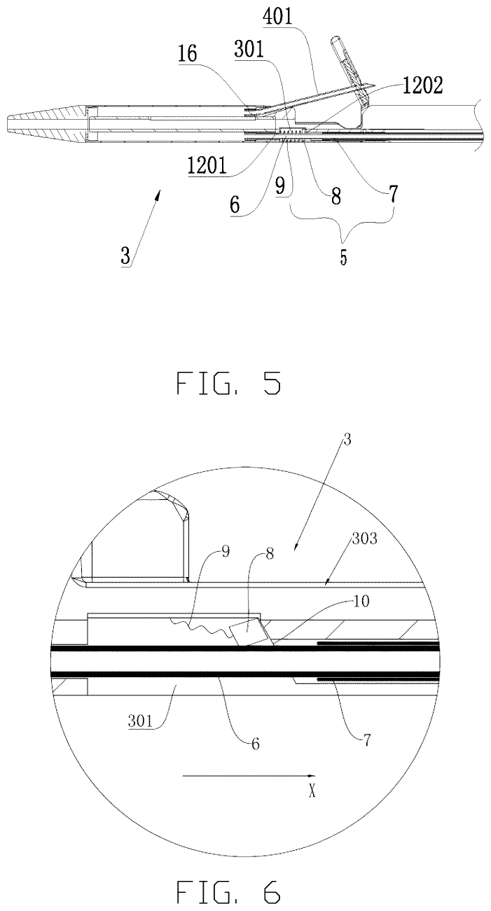

Referring to FIGS. 3 to 5, a puncture locking device is provided according to the present application, which includes a suture head 3. A suture assembly 4 and a locking structure 5 are arranged inside the suture head 3. The suture assembly 4 includes a suture needle 401 and a push-pull guiding member 6 operatively coupled to the suture needle 401. The locking structure 5 includes an elastic locking assembly and an unlocking member 7. The elastic locking assembly includes a locking portion 8 for restricting a movement of the push-pull guiding member 6 and an elastic portion 9 for providing a locking force to the locking portion 8. In a locked state, the elastic portion 9 applies a force to the locking portion 8, causing the locking portion 8 to be tilted and abut against the push-pull guiding member 6, thereby restricting the movement of the push-pull guiding member 6 along a first direction X. The first direction X is the moving direction of the push-pull guiding member 6 when the push-pull guiding member 6 drives the suture needle 401 to penetrate out of the suture head 3. In an unlocked state, the unlocking member 7 moves within the suture head 3 to counterbalance the force applied by the elastic portion 9 to the locking portion 8, thereby unlocking the push-pull guiding member 6.

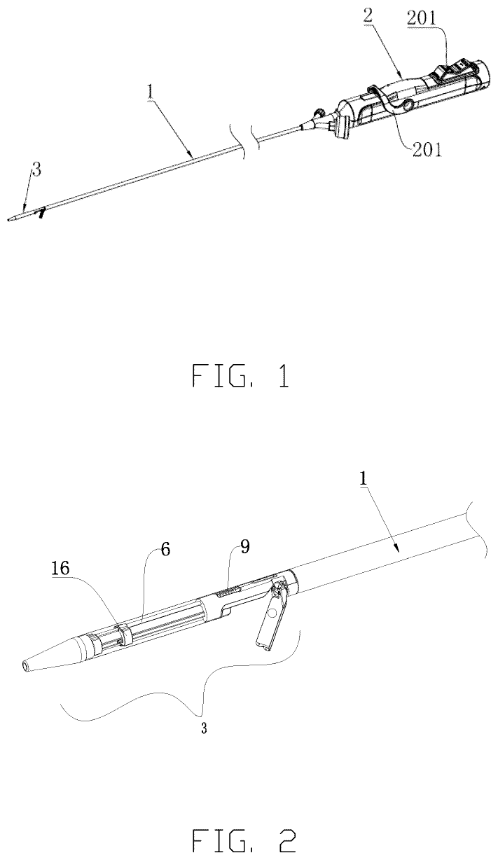

The puncture locking device of the present application may be specifically used in a suture apparatus. The puncture locking device in combination with a specific suture apparatus will be described as follows. Referring to FIGS. 1 and 2, the suture apparatus includes a catheter 1 and a handheld operation portion 2 disposed at a proximal end of the catheter 1. The suture head 3 is disposed at a distal end of the catheter 1. The push-pull guiding member 6 extends through the catheter 1 and may reciprocally move along an axial direction of the catheter 1 to drive the suture needle 401 to move. The unlocking member 7 extends through the catheter 1 and may move along the axial direction of the catheter 1. Both of the push-pull guiding member 6 and the unlocking member 7 are connected to the handheld operation portion 2. The handheld operation portion 2 can control the push-pull guiding member 6 and the unlocking member 7 to move.

It can be understood that the suture head 3 is disposed at the distal end of the catheter 1, and the handheld operating portion 2 is disposed at the proximal end of the catheter 1, thereby facilitating the operation of the suture apparatus when the suture apparatus is intervened into the human body. The push-pull guiding member 6 is operatively coupled to the suture needle 401 and connected to the handheld operation portion 2, so that the movement of the suture needle 401 can be simply controlled by manipulating the handheld operation portion 2. The push-pull guiding member 6 is a push-pull tube structure used in medical devices. The push-pull guiding member 6 can be directly or indirectly connected to the suture needle 401, so long as the push-pull guiding member 6 can drive the suture needle 401 to protrude from or retract back to the suture head 3.

The locking structure 5 is also disposed within the suture head 3, and is located at the distal end of the catheter 1. The locking structure 5 prevents the suture needle 401 from protruding from the suture head 3 by directly restricting the position of the push-pull guiding member 6 near the suture needle 401, making the locking structure relatively stable. Furthermore, by disposing the locking structure 5 directly within the suture head 3 and close to the suture needle 401, the locking structure 5 can quickly lock the push-pull guiding member 6 without any loss of force transmission therebetween, thereby improving the accuracy and stability of the locking. Because interventional suture devices typically have elongated and flexible catheters to accommodate tortuous cardiovascular anatomical structure, thereby may be a loss in the force transmission through such flexible catheter structures. Compared to arranging the locking structure 5 within the handheld operation portion 2, the present application arranges the locking structure 5 within the suture head 3, thereby avoiding the problems of delayed locking and low locking accuracy caused by force transmission loss.

In the locked state, the locking structure 5 utilizes the elastic portion 9 to apply a force to the locking portion 8, causing the locking portion 8 to be tilted and abut against the push-pull guiding member 6, thereby restricting the position of the push-pull guiding member 6. It can be understood that when the locking portion 8 obliquely abuts against the push-pull guiding member 6, a frictional force is generated between the locking portion 8 and the push-pull guiding member 6 that restricts the movement of the push-pull guiding member 6. The elastic force of the elastic portion 9 provides a locking force to the locking portion 8 such that the frictional force between the locking portion 8 and the push-pull guiding member 6 is sufficient to restrict the movement of the push-pull guiding member 6.

Furthermore, the locking portion 8 obliquely abuts against the push-pull guiding member 6. The oblique direction of the locking portion 8 is relative to the first direction X. The purpose is that the oblique abutment can better restrict the movement of the push-pull guiding member 6 along the first direction X, thereby better restricting the suture needle 401 from penetrating out of the suture head 3 and making the locking more secure.

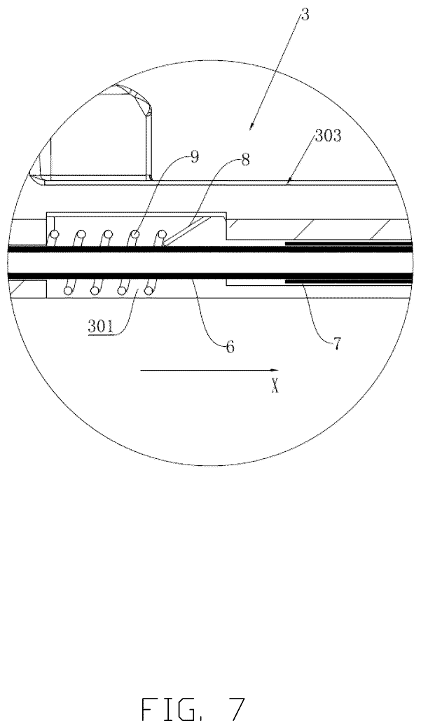

Referring to FIGS. 7, 8, and 9, the first direction X is specifically the moving direction of the push-pull guiding member 6 within the suture head 3 when the push-pull guiding member 6 drives the suture needle 401 to penetrate out of the suture head 3. Restricting the movement of the push-pull guiding member 6 along the first direction X locks the suture needle 401 to prevent the suture needle 401 from protruding from the suture head 3, thereby reducing or avoiding any damage to the of heart tissue caused by the pre-triggering of the suture needle 401 under misoperation.

Depending on the setting of the penetrating direction of the suture needle 401, the first direction may be a direction from the proximal end of the catheter 1 toward the distal end of the catheter 1, or from the distal end of the catheter 1 toward the proximal end of the catheter 1. In this embodiment, the penetrating direction of the suture needle 401 is a direction from the distal end of the catheter 1 toward the proximal end of the catheter 1, and the first direction is the direction X shown in FIGS. 7, 8, and 9.

The connection method and specific structure of the elastic portion 9 and the locking portion 8 are not limited, so long as the elastic portion 9 can act on the locking portion 8 to cause the locking portion 8 to be tilted and abut against the push-pull guiding member 6. The locking portion 8 may be a locking block, a locking hole, or other structures. The elastic portion 9 may be a spring, an elastic sheet, or other structures.

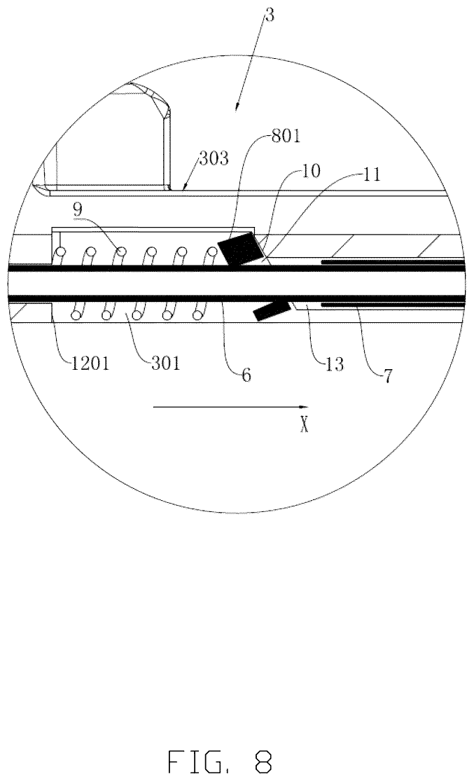

Referring to FIGS. 6 to 8, in some embodiments, the locking portion 8 is a locking block. Referring to FIGS. 6 and 8, an oblique wall structure may be provided inside the suture head 3. The elastic portion 9 acts on the locking portion 8, causing the locking portion 8 to contact the oblique wall structure. This contact causes the locking portion 8 to be tilted and abut against the push-pull guiding member 6, thereby restricting the movement of the push-pull guiding member 6 along the first direction X. Furthermore, when the locking portion 8 is the locking block, the connection and cooperation relationship of the locking portion 8 and the push-pull guiding member 6 are not limited. Referring to FIG. 8, the push-pull guiding member 6 may extend through the locking portion 8, and the locking portion 8 is tilted to abut against the push-pull guiding member 6, thereby restricting the position of the push-pull guiding member 6. Referring to FIG. 6, the locking portion 8 may be disposed only on one side of the push-pull guiding member 6. When the locking portion 8 is tilted, the locking portion 8 abuts against the corresponding side of the push-pull guiding member 6 to restrict the position of the push-pull guiding member 6.

Referring to FIG. 7, in some embodiments, the oblique wall structure is omitted from the suture head 3. Instead, one end of the locking portion 8 is directly hinged to the inner wall of the suture head 3, and the elastic portion 9 acts directly on the locking portion 8 to cause the locking portion 8 to pivot and tilt against the push-pull guiding member 6, which can also restrict the position of the push-pull guiding member 6.

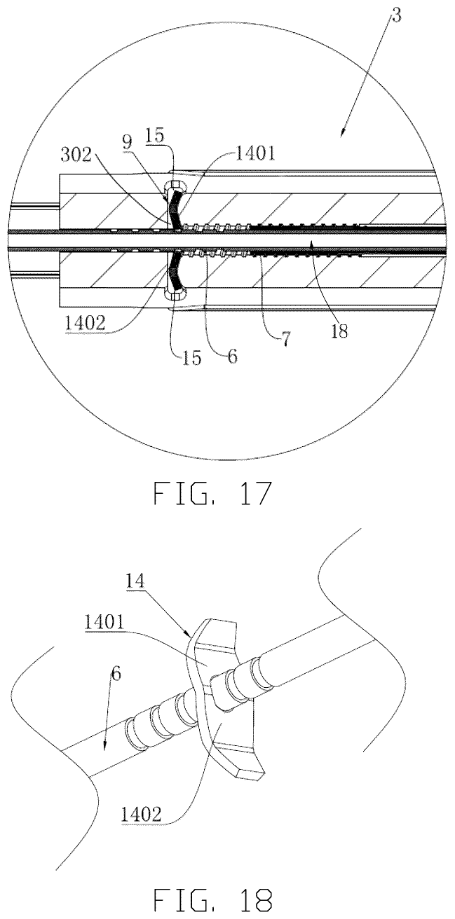

Referring to FIGS. 16 to 21, in some embodiments, the locking portion 8 may be a locking hole, and the push-pull guiding member 6 extends through the locking hole. The elastic portion 9 is elastic deformed such as being bent at the locking hole, to adjust the size of the locking hole. In the locked state, the locking hole is in an inclined form such that the hole wall of the locking hole abuts against the push-pull guiding member 6, which can also restrict the movement of the push-pull guiding member 6 along the first direction X.

The unlocking member 7 extends through the catheter 1 and is capable of moving along the axial direction of the catheter 1. The unlocking member 7 is connected to the handheld operation portion 2, whereby the manipulation of the handheld operation portion 2 controls the movement of the unlocking member 7.

In the unlocked state, the unlocking member 7 is capable of moving along the axial direction of the catheter 1 to counterbalance the force applied on the locking portion 8 by the elastic portion 9, thereby unlocking the push-pull guiding member 6. The function of the unlocking member 7 is to counteract the locking force applied onto the locking portion 8 by the elastic portion 9, causing the locking portion 8 to separate from the push-pull guiding member 6 and thus unlock the push-pull guiding member 6. Here, the separation between the locking portion 8 and the push-pull guiding member 6 does not necessarily mean that the two elements are in a state of complete physical disengagement. Rather, it indicates that the frictional force between them is negligible or insufficient to restrict the movement of the push-pull guiding member 6. The locking portion 8 and the push-pull guiding member 6 may be in contact or not in contact with each other.

Furthermore, the acting target and acting manner of the unlocking member 7 are not unique. For example, the unlocking member 7 can directly act on the locking portion 8, either by pushing or pulling the locking portion 8 to counterbalance the force from the elastic portion 9. Alternatively, the unlocking member 7 can directly act on the elastic portion 9 to counterbalance the force of the elastic portion 9.

The puncture locking device of the present application is capable of locking the suture needle 401, and avoiding the risk of misoperation by providing the locking structure 5 within the suture head 3 that can limit and lock the push-pull guiding member 6. When the puncture locking device is applied within the suture device, the suture head 3 is disposed at the distal end of the catheter 1, and the locking structure 5 is disposed within the suture head 3 and near the suture needle 401, meaning the locking structure 5 is located at the distal end of the catheter 1, thereby achieving more stable locking. The locking portion 8 is tilted and abut against the push-pull guiding member 6 for limiting the position of the push-pull guiding member 6. The locking structure is simple and reliable, providing effective position limiting function. Moreover, the locking structure 5 is compact in structure and can be directly disposed within the suture head 3 without increasing the overall size of the device.

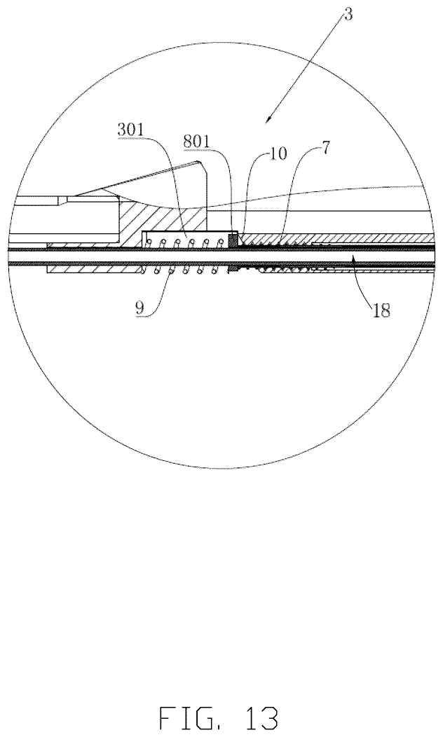

Referring to FIGS. 8 to 15, in some embodiments, an inclined abutting wall 10 is provided within the suture head 3, and the locking portion 8 includes a locking block 801. In the locked state, the elastic portion 9 acts on the locking block 801 to abut against the inclined abutting wall 10, such that the locking block 801 to deflects and tilts into abutment with the push-pull guiding member 6.

Under the elastic force of the elastic portion 9, the locking block 801 tends to slide along an inclined surface of the inclined abutting wall 10, causing the locking block 801 to continuously press and abut against the push-pull guiding member 6 in a direction perpendicular to the axial direction of the push-pull guiding member 6, thereby enabling the locking block 801 to restrict the movement of the push-pull guiding member 6.

Furthermore, the inclined surface of the inclined abutting wall 10 is inclined relative to the first direction X. That is, an angle between the inclination direction of the inclined abutting wall 10 and the first direction X is an obtuse angle, causing the locking block 801 to always have a tendency to slide down the inclined surface of the inclined abutting wall 10 and abut against the push-pull guiding member 6 under the elastic force of the elastic portion 9.

By adopting the above technical solution, the inclined abutting wall 10 allows the elastic force of the elastic portion 9 to quickly act on the locking block 801 to cause the locking block 801 to change to an inclined state, causing the locking block 801 to be titled and abut against the push-pull guiding member 6. The position limiting action occurs very rapidly, making the locking structure 5 to quickly lock the push-pull guiding member 6.

Referring to FIGS. 8 to 15, in some embodiments, the locking block 801 has a via hole 11. The push-pull guiding member 6 extends through the via hole 11, and the outer wall of the push-pull guiding member 6 and the inner wall of the via hole 11 are engaged in a clearance fit. The locking block 801 is inclined so that the push-pull guiding member 6 abuts against the inner wall of the via hole 11 of the locking block 801.

Since the outer wall of the push-pull guiding member 6 is engaged with the inner wall of the via hole 11 in a clearance fit manner, when the locking block 801 is not inclined and abutting against the push-pull guiding member 6, the push-pull guiding member 6 can move within the via hole 11, and the locking block 801 does not limit the push-pull guiding member 6 at this time.

Referring to FIG. 8, when the locking block 801 is inclined and abuts against the push-pull guiding member 6 for limiting the position of the push-pull guiding member 6, two opposite inner walls of the via hole 11 of the locking block 801 can abut against the push-pull guiding member 6 and limit the position of the push-pull guiding member 6, thereby providing a dual abutting and position limiting effect, making the limiting structure more stable. Moreover, with this limiting structure, when the push-pull guiding member 6 is subjected to a force moving in the first direction X, the locking block 801 tends to press more closely against the inclined abutting wall 10, thereby causing the locking block 801 and the push-pull guiding member 6 to abut against each other more tightly, thereby increasing the locking force therebetween.

By adopting the above technical solution, when the locking block 801 is titled, two opposite sides of the via hole 11 of the locking block 801 can abut against the push-pull guiding member 6 and limit the position of the push-pull guiding member 6, thereby achieving a better position limiting structure. Furthermore, with this limiting structure, the more the push-pull guiding member 6 moves in the first direction X, the more the locking block 801 tends to press closely against the inclined abutting wall 10, thereby increasing the locking force between the locking block 801 and the push-pull guiding member 6, making the locking principle and effect more stable and reliable.

Referring to FIGS. 3 to 5 and 8 to 15, in some embodiments, the suture head 3 has a first accommodating cavity 301 for accommodating an elastic locking assembly. The inclined abutting wall 10 is located within the first accommodating cavity 301, and the push-pull guiding member 6 passes through the first accommodating cavity 301. The elastic portion 9 and the locking block 801 are separately disposed. One end of the elastic portion 9 abuts against an inner wall of the first accommodating cavity 301, and the other end of the elastic portion 9 abuts against the locking block 801.

In at least one embodiment, the suture head 3 includes a housing 303. The first accommodating cavity 301 for accommodating the elastic locking assembly is provided within the housing 303, and the push-pull guiding member 6 passes through the first accommodating cavity 301. The elastic portion 9 and the locking block 801 are also disposed within the first accommodating cavity 301, and the elastic portion 9 and the locking block 801 are separated from each other. One end of the elastic portion 9 abuts against the wall of the first accommodating cavity 301, and the other end of the elastic portion 9 abuts against the locking block 801. The locking structure is compact in structure and can effectively act on the push-pull guiding member 6.

The inclined abutting wall 10 may be disposed at any position within the first accommodating cavity 301. The inclined abutting wall 10 may be disposed on an end surface of the first accommodating cavity 301. Alternatively, a protrusion structure may be additionally provided within the first accommodating cavity 301, and the inclined abutting wall 10 is provided on the protrusion structure.

In the above technical solution, the first accommodating cavity 301 allows for better arrangement of the locking structure 5. It is only necessary to place the elastic portion 9 and the locking block 801 within the first accommodating cavity 301, making the installation simple, saving assembly steps, and improving assembly efficiency.

Referring to FIGS. 3 to 5 and 8 to 15, in some embodiments, along the first direction X, the first accommodating cavity 301 has a first end surface 1201 closer to the suture needle 401 and a second end surface 1202 farther from the suture needle 401. The second end surface 1202 is inclined to form the inclined abutting wall 10.

In the above technical solution, directly inclining the second end surface 1202 to form the inclined abutting wall 10 makes the design of the inclined wall more convenient, without excessively occupying the internal space of the suture head 3, so that the puncture locking device has a smaller volume that occupies less space during surgery, thereby achieving less trauma.

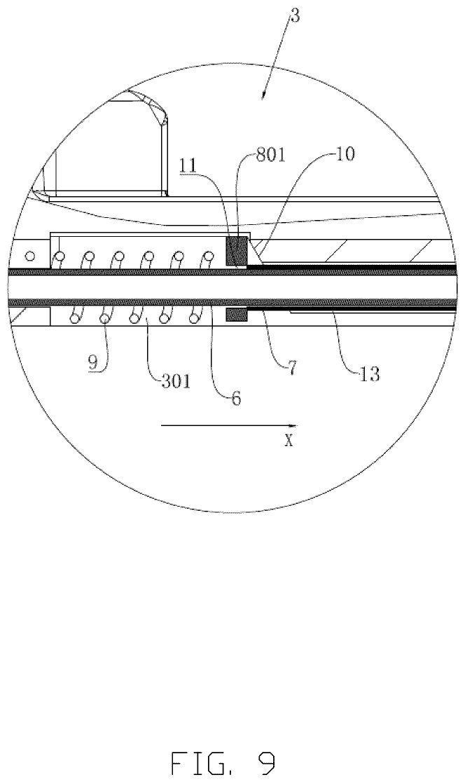

Referring to FIGS. 8 and 9, in some embodiments, the elastic portion 9 may be a spring. The spring is disposed within the first accommodating cavity 301 along the axial direction of the push-pull guiding member 6, and the push-pull guiding member 6 passes through the spring.

The spring is arranged in the first accommodating cavity 301 along the axial direction of the push-pull guiding member 6, and the spring is sleeved on the push-pull guide element 6. The spring is in a pre-compressed state, enabling the spring to apply a pushing force to the locking block 801, causing the locking block 801 to abut against the inclined abutting wall 10. Setting the elastic portion 9 as a spring provides a simple structure that can accommodate a certain assembly tolerance, and provides timely locking feedback, making the locking more stable and accurate.

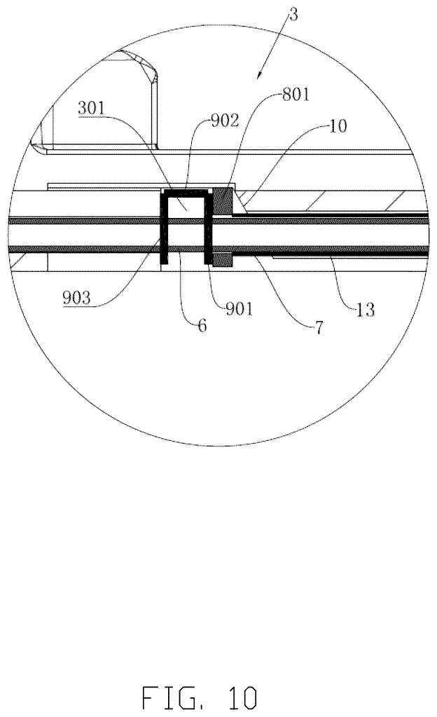

Referring to FIGS. 10, 11, 14, and 15, in some embodiments, the elastic portion 9 may be a bent elastic sheet. The elastic sheet is disposed within the first accommodating cavity 301 along the length direction of the push-pull guiding member 6. The elastic sheet includes at least one first sub-plate 901 that is close to the locking block 801 and elastically acts on the locking block 801.

The first sub-plate 901 is in a pre-compressed state, enabling the first sub-plate 901 to apply a pushing force to the locking block 801, causing the locking block 801 to abut against the inclined abutting wall 10.

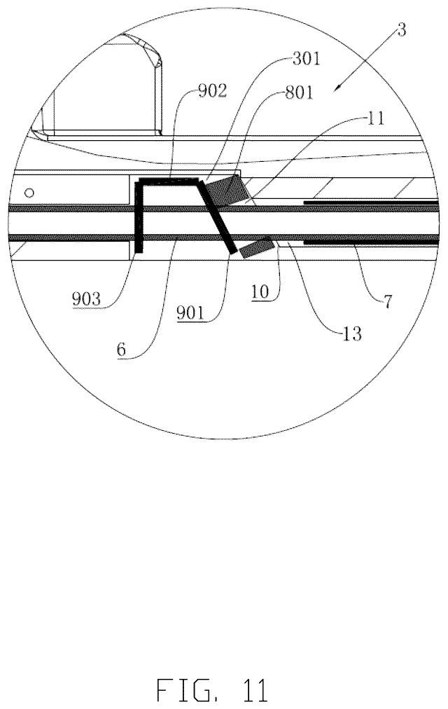

The specific bent structure of the elastic sheet is not limited, and the elastic sheet may have a two-segment or multi-segment structure. In this embodiment, referring to FIGS. 10 and 11, the elastic sheet has a three-segment structure. The elastic sheet includes the first sub-plate 901, a second sub-plate 902 connected to the first sub-plate 901, and a third sub-plate 903 connected to an end of the second sub-plate 902 away from the first sub-plate 901. The second sub-plate 902 is parallel to the push-pull guiding member 6, and the third sub-plate 903 is abutting against the first end surface 1201. The first sub-plate 901 has a tendency to be titled toward the first direction X to apply a pushing force to the locking block 801. The push-pull guiding member 6 passes through the first sub-plate 901 and the third sub-plate 903, and the push-pull guiding member 6 can move relative to the elastic portion 9.

Referring to FIGS. 8, 9, 12, and 13, in some embodiments, the unlocking member 7 extends through the suture head 3. An end of the unlocking member 7 can push the locking block 801 to separate the locking block 801 from the push-pull guiding member 6.

In at least one embodiment, a through hole 13 communicating with the first accommodating cavity 301 is provided in the housing 303. The through hole 13 penetrates to the inclined abutting wall 10, and the unlocking member 7 passes through the through hole 13. The unlocking member 7 can thus enter the first accommodating cavity 301 through the through hole 13. In the present application, the first direction X is the direction from the distal end to the proximal end of the catheter 1. The end of the unlocking member 7 can push the locking block 801 to move in a direction opposite to the first direction X to balance the force applied onto the locking block 801 by the elastic portion 9, causing the locking block 801 to gradually return from the inclined state to an upright state and separate from the push-pull guiding member 6, thereby unlocking the push-pull guiding member 6 from the locking block 801.

Here, separation between the locking portion 8 and the push-pull guiding member 6 does not necessarily mean a complete positional separation, but instead indicates a state where the friction therebetween is negligible and does not restrict the movement of the push-pull guiding member 6. The locking portion 8 and the push-pull guiding member 6 may be in contact or not in contact with each other.

The unlocking member 7 is a push tube structure in medical devices. Moreover, the diameter of the unlocking member 7 is larger than the diameter of the via hole 11 in the locking block 801, allowing the unlocking member 7 to directly push the locking block 801 to move.

Referring to FIGS. 18 to 21, in some embodiments, the elastic portion 9 includes an elastic main body 14 disposed along a direction perpendicular to the axial direction of the push-pull guiding member 6. The locking portion 8 is a locking hole 802 provided on the elastic main body 14, and the push-pull guiding member 6 passes through the locking hole 802. The suture head 3 has a second accommodating cavity 302 for accommodating the elastic main body 14, and an end of the elastic main body 14 distal from the push-pull guiding member 6 is positionally limited at a wall of the second accommodating cavity 302. The elastic main body 14 includes a first bent segment 1401 and a second bent segment 1402 arranged at an angle to each other. The locking hole 802 is located at a junction of the first bent segment 1401 and the second bent segment 1402. The unlocking member 7 is configured to act upon the junction of the first bent segment 1401 and the second bent segment 1402, so that the junction is elastically deformed to adjust the size of the locking hole 802. In the locked state, an inner wall of the locking hole 802 abuts against the push-pull guiding member 6. In the unlocked state, the inner wall of the locking hole 802 is separated from the push-pull guiding member 6.

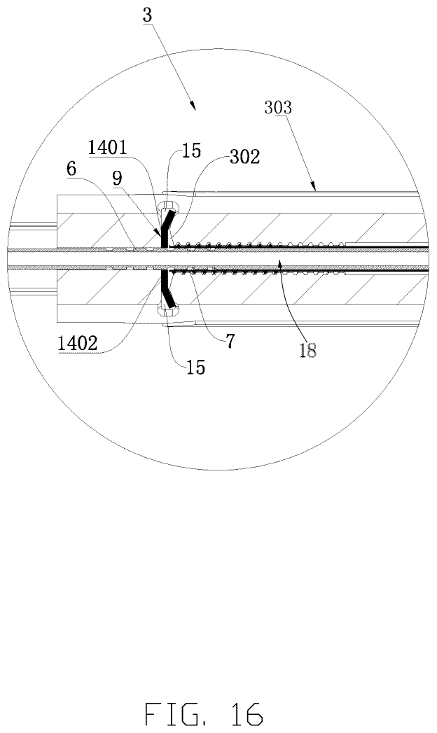

The elastic main body 14 is disposed within the second accommodating cavity 302 along the direction perpendicular to the axial direction of the push-pull guiding member 6. Referring to FIGS. 16 and 17, a groove 15 is provided in the cavity wall at a position corresponding to the elastic main body 14 within the second accommodating cavity 302. The end of the elastic main body 14 is engaged within this groove 15, thereby restricting movement of the elastic main body 14 along the axial direction of the push-pull guiding member 6.

Referring to FIGS. 18 to 21, the locking hole 802 is located at the junction of the first bent segment 1401 and the second bent segment 1402. Consequently, when a force is applied to the junction of the first bent segment 1401 and the second bent segment 1402 and the junction is elastically deformed, the aperture size of the locking hole 802 in the direction perpendicular to the axial direction of the push-pull guiding member 6 changes accordingly.

Referring to FIGS. 17 to 21, when no external force is applied to the elastic main body 14, the first bent segment 1401 and the second bent segment 1402 form an angle, which is significantly less than 180°. In this state, the aperture size of the locking hole 802 in the direction perpendicular to the axial direction of the push-pull guiding member 6 is in a compressed state. That is, the portion of the locking hole 802 located on the first bent segment 1401 and the portion located on the second bent segment 1402 are both inclined, causing the wall of the locking hole 802 to abut against the push-pull guiding member 6, thereby locking the push-pull guiding member 6.

The size of the locking hole 802 is adjusted after the unlocking member 7 acts on the junction of the first bent segment 1401 and the second bent segment 1402 and the junction is elastically deformed. The manner in which the unlocking member 7 acts upon the elastic main body 14 is not limited. For example, the unlocking member 7 may act on the elastic main body 14 either by a pushing or a pulling method.

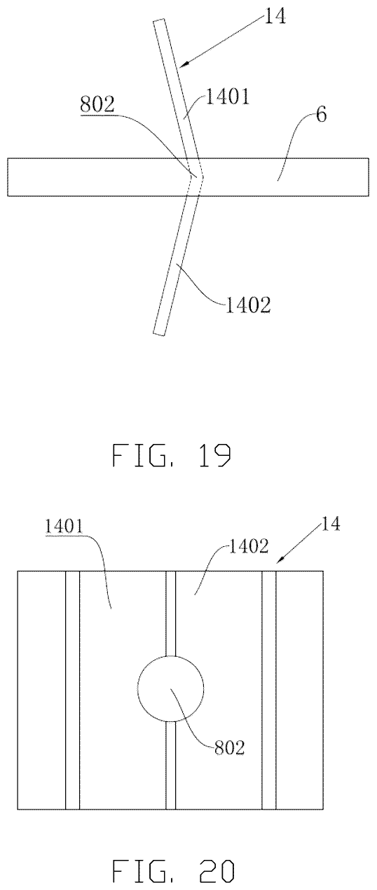

Referring to FIGS. 20 and 21, when the unlocking member 7 acts on the junction of the first bent segment 1401 and the second bent segment 1402 and the junction is elastically deformed, the angle between the first bent segment 1401 and the second bent segment 1402 tends to be 180°. This causes the aperture size of the locking hole 802 in the direction perpendicular to the axial direction of the push-pull guiding member 6 to increase, so that the inner wall of the locking hole 802 and the outer wall of the push-pull guiding member 6 are engaged in a clearance fit manner, thereby separating the locking hole wall from the push-pull guiding member 6 and unlocking the push-pull guiding member 6.

In the above technical solution, the locking portion 8 is the locking hole 802 directly provided on the elastic main body 14, making the elastic portion 9 and the locking portion 8 to be an integrated structure. The unlocking member 7 can cause the junction of the first bent segment 1401 and the second bent segment 1402 to be elastically deformed to adjust the size of the locking hole 802, achieving locking and unlocking of the push-pull guiding member 6. This locking adjustment method is simple and effective, the overall structure is more streamlined, and spatial occupancy is reduced.

Referring to FIGS. 16 and 17, the unlocking member 7 is disposed through the suture head 3. An end of the unlocking member 7 can push the elastic main body 14 to separate the locking hole 802 from the push-pull guiding member 6. In at least one embodiment, the unlocking member 7 is a metal push tube structure in medical devices, and the diameter of the unlocking member 7 is greater than the maximum diameter of the locking hole 802. The end of the unlocking member 7 can directly push against the junction of the first bent segment 1401 and the second bent segment 1402 to cause elastic deformation of the elastic main body 14, thereby adjusting the size of the locking hole 802.

Separation between the locking hole 802 and the push-pull guiding member 6 does not necessarily denote a complete disengagement, but instead indicates a state where the inner wall of the locking hole 802 and the outer wall of the push-pull guiding member 6 are engaged in a clearance fit manner, thereby allowing the push-pull guiding member 6 to move. In this state, the locking hole 802 and the push-pull guiding member 6 may be in contact or not in contact with each other.

Referring to FIGS. 4 to 17, in some embodiments, the unlocking member 7 is a metal flexible tube structure. The unlocking member 7 is sleeved over the push-pull guiding member 6, and the unlocking member 7 and the push-pull guiding member 6 move independently of each other.

In at least one embodiment, the unlocking member 7 is a metal flexible tube used in medical devices, providing a rigid structure at its end for effectively applying a pushing force.

Referring to FIG. 9, when the locking portion 8 is the locking block 801, the end of the unlocking member 7 can directly push against the locking block 801, thereby directly counteracting the elastic force applied by the elastic component 9 to the locking block 801 and separating the locking block 801 from the push-pull guiding member 6. The pushing force of the unlocking member 7 is directly applied to the locking block 801, avoiding force loss.

Referring to FIG. 16, when the locking portion 8 is the locking hole 802 on the elastic main body 14, the end of the unlocking member 7 can directly push against the elastic main body 14.

Sleeving the unlocking member 7 over the push-pull guiding member 6 allows for a more rational structural layout and reduces the occupation of internal space. Furthermore, the unlocking member 7 and the push-pull guiding member 6 move independently, meaning the movement of the unlocking member 7 does not interfere with the push-pull guiding member 6, and vice versa.

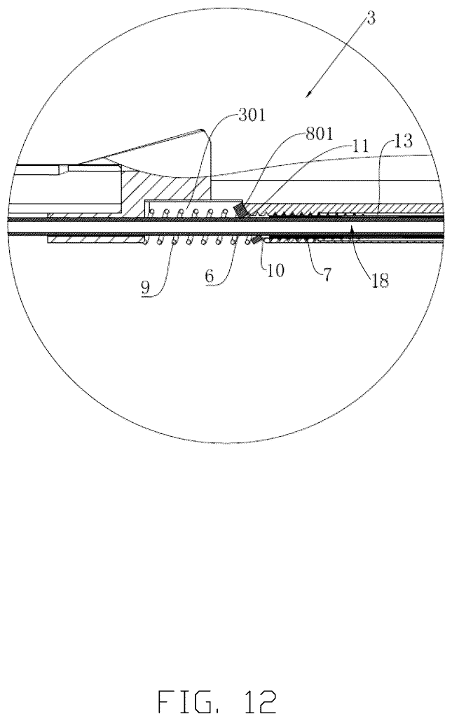

Referring to FIGS. 12, 13, 16, and 17, in some embodiments, a threaded hole 18 is provided within the suture head 3, and the unlocking member 7 is threadedly engaged within the threaded hole 18.

The unlocking member 7 is a metal flexible tube, allowing for the addition of a threaded structure on the unlocking member 7, so that the unlocking member 7 can threadedly engage with the threaded hole. Using a threaded engagement allows for precise control of the movement distance of the unlocking member 7. Consequently, simply rotating the unlocking member 7 by a certain number of turns can control the switching of the locking portion 8 between the locked and unlocked states, thereby providing better locking control.

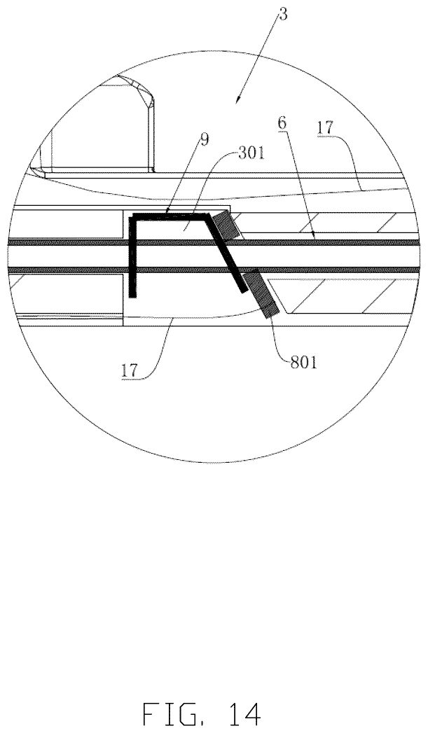

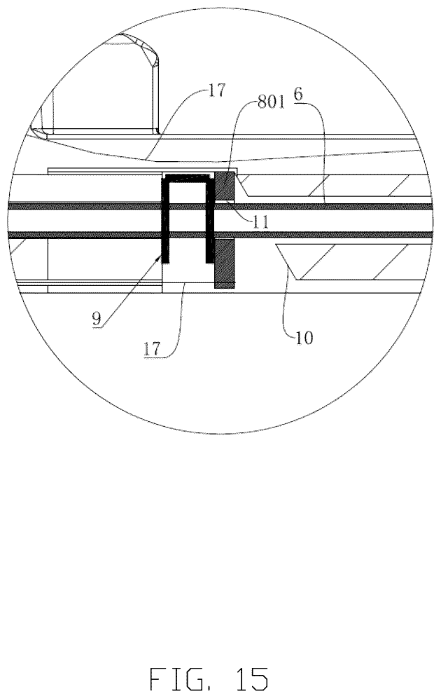

Referring to FIGS. 14 and 15, in some embodiments, the unlocking member 7 is a pull wire 17. The locking portion 8 separates from the push-pull guiding member 6 under a pulling force of the pull wire 17.

The pull wire 17 provides the pulling force, thereby achieving a simpler structure.

Referring to FIGS. 14 and 15, when the locking portion 8 is the locking block 801, the pull wire 17 can be connected to the locking block 801 or to the elastic portion 9. By pulling the locking block 801 or the elastic portion 9 counteracts the elasticity of the elastic portion 9, the locking block 801 is separated from the push-pull guiding member 6, thereby unlocking the push-pull guiding member 6.

Referring to FIGS. 16 to 19, when the locking portion 8 is the locking hole 802 on the elastic main body 14, the pull wire 17 is connected to the elastic main body 14. By pulling the pull wire 17 to cause elastic deformation of the first bent segment 1401 and the second bent segment 1402, the locking hole 802 is separated from the push-pull guiding member 6, thereby unlocking the push-pull guiding member 6.

Referring to FIGS. 2 and 3, in some embodiments, the suture assembly 4 further includes a slider 16 slidably disposed within the suture head 3. The suture needle 401 and the push-pull guiding member 6 are respectively connected to the slider 16.

In at least one embodiment, one end of the suture needle 401 is connected to the slider 16, and one end of the push-pull guiding member 6 is connected to the slider 16. Consequently, the movement of the push-pull guiding member 6 drives the slider 16 to slide, causing the suture needle 401 to move, enabling the push-pull guiding member 6 to control the movement of the suture needle 401 more smoothly.

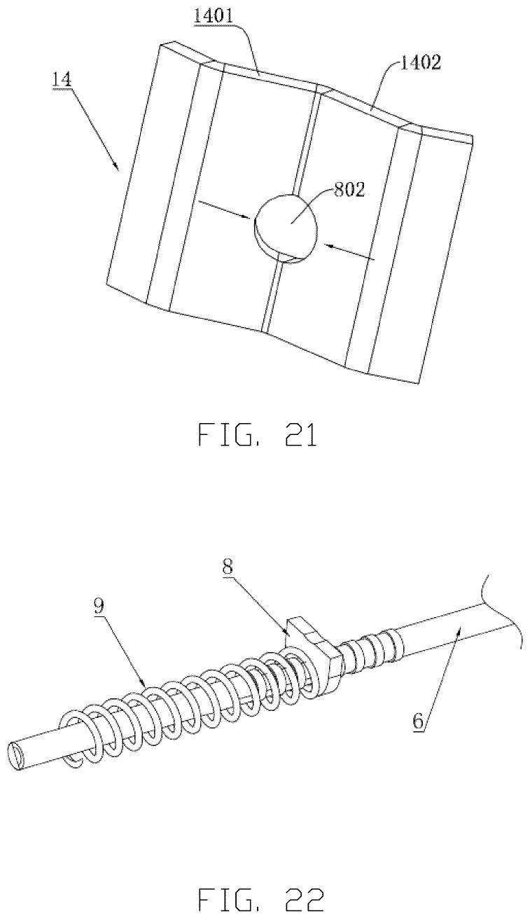

Referring to FIG. 22, an anti-slip structure is further provided on the push-pull guiding member 6.

The specific form of the anti-slip structure is not limited. The anti-slip structure may be protrusions spaced apart from each other on the push-pull guiding member 6 or a textured pattern formed on the push-pull guiding member 6. When the locking portion 8 obliquely abuts against the push-pull guiding member 6, the end of the locking portion 8 is in contact with the anti-slip structure, thereby increasing friction between the push-pull guiding member 6 and the locking portion 8 and enhancing the limiting and locking effect.

Referring to FIGS. 1, 2, and 23, the present application also provides a suture apparatus. The suture apparatus includes the catheter 1, the handheld operation portion 2 disposed at a proximal end of the catheter 1, and the aforementioned puncture locking device. The suture head 3 is disposed at a distal end of the catheter 1. The push-pull guiding member 6 extends through the catheter 1 and is configured to reciprocate along the axial direction of the catheter 1. The unlocking member 7 is disposed through the catheter 1 and is configured to move along the axial direction of the catheter 1. Both the push-pull guiding member 6 and the unlocking member 7 are connected to the handheld operation portion 2. The handheld operation portion 2 is configured to control movement of the push-pull guiding member 6 and the unlocking member 7.

It is understandable that the locking structure 5 can be controlled to lock or unlock the suture needle 401 by manipulating the handheld operation portion 2, thereby enabling proximal adjustment and operation.

Although the locking or unlocking operation for the suture needle 401 is performed via the proximal handheld operation portion 2, the locking structure 5 is disposed within the suture head 3, i.e., the locking structure 5 is located at the distal end of the catheter 1. The locking structure 5 restricts the penetration of the suture needle 401 by providing a limiting and locking force to the push-pull guiding member 6 near the suture needle 401, effectively locking the distal push-pull guiding member 6.

In at least one embodiment, referring to FIG. 24, during actual operation of the suture apparatus of the present application, the suture head 3 is intervened into a human body via the catheter 1. To accommodate the tortuous cardiovascular anatomy, the catheter 1 is constructed as an elongated and flexible structure. This flexible catheter construction results in force transmission losses, such that relying solely on a locking force applied at the handheld operation portion 2 would be insufficient to completely lock the movement of the distal suture needle 401. Therefore, the present application disposes the locking structure 5 within the suture head 3, providing a locking force directly to the distal push-pull guiding member 6. This ensures complete locking of the movement of the distal suture needle 401, making the locking structure more stable.

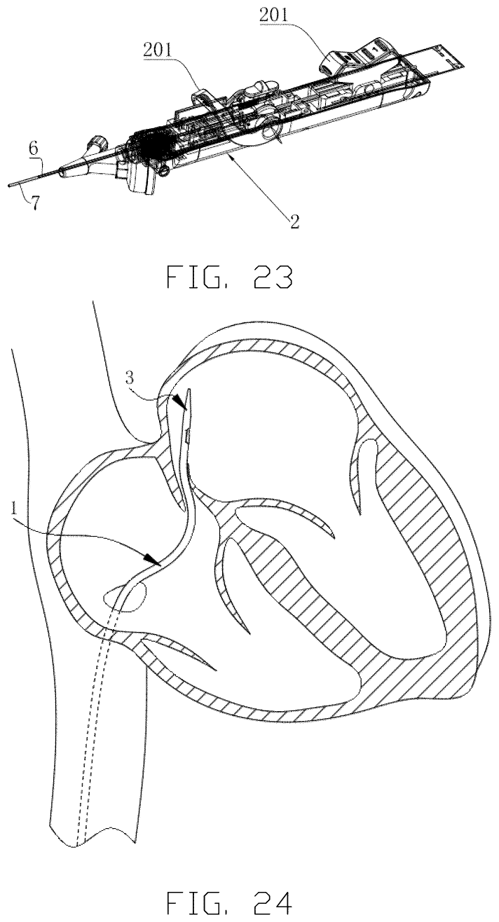

Referring to FIGS. 1 and 23, the operating handpiece 2 is provided with an adjustment switch 201 for regulating the movement of the push-pull guiding member 6 and the unlocking member 7.

Both of the push-pull guiding member 6 and the unlocking member 7 are connected to the handheld operation portion 2 via the catheter 1. The handheld operation portion 2 is provided with the adjustment switch 201 for regulating the movement of the push-pull guiding member 6 and the unlocking member 7. The specific form of the adjustment switch 201 is not limited, as long as the adjustment switch 201 can control the movement of the push-pull guiding member 6 and the unlocking member 7. The adjustment switch 201 may be a trigger mechanism, a slider mechanism, a button mechanism, a knob mechanism, or a pull-and-rotate lever structure.

In the aforementioned technical solutions, the locking structure 5, which is located at the distal end of the catheter 1 within the suture head 3, can directly lock the suture needle 401. This enables rapid and timely locking without requiring feedback time, while also providing a more stable and precise locking effect. When the suture needle 401 needs to be extended for puncture, the operator can control the unlocking member 7 to unlock the push-pull guiding member 6 by operating at the proximal end of the catheter 1, allowing the suture needle 401 to extend smoothly. This adjustment is convenient, accurate, and reliable.

The above description are exemplarily embodiments of the present application but not considered as limiting the present application. For a person skilled in the art, modifications and changes may be made in the detail. Within the principles of the present disclosure, any modification, equivalent substitution, or change shall be included within the scope of the appended claims.

What is claimed is:

1. A puncture locking device comprising a suture head, a suture assembly and a locking structure arranged inside the suture head,
the suture assembly comprising a suture needle and a push-pull guiding member operatively coupled to the suture needle; and
the locking structure comprising an elastic locking assembly and an unlocking member, the elastic locking assembly comprising a locking portion configured to limit a position of the push-pull guiding member and an elastic portion configured to elastically deformed against the locking portion;
wherein in a locked state, the elastic portion elastically deforms against the locking portion, the locking portion is tilted by the elastic portion and abuts against the push-pull guiding member, thereby restricting a movement of the push-pull guiding member along a first direction, and the first direction is a moving direction of the push-pull guiding member in response to the push-pull guiding member drives the suture needle to penetrate out of the suture head; and
wherein in an unlocked state, the unlocking member moves within the suture head to release the locking portion from tilting and abutting against the push-pull guiding member.

2. The puncture locking device according to claim 1, wherein an inclined abutting wall is arranged within the suture head, and the locking portion comprises a locking block; in the locked state, the elastic portion elastically deforms against the locking block to cause the locking block to abut against the inclined abutting wall, such that the locking block is titled and abuts against the push-pull guiding member.

3. The puncture locking device according to claim 2, wherein the locking block defines a via hole, the push-pull guiding member extends through the via hole, an outer wall of the push-pull guiding member and an inner wall of the via hole are engaged in a clearance fit manner; in the locked state, the locking block is titled to cause the push-pull guiding member to abut against the inner wall of the via hole of the locking block.

4. The puncture locking device according to claim 2, wherein the suture head defines a first accommodating cavity configured for accommodating the elastic locking assembly, and the inclined abutting wall is located within the first accommodating cavity; the push-pull guiding member extends through the first accommodating cavity; the elastic portion and the locking block are separated from each other, one end of the elastic portion abuts against an inner wall of the first accommodating cavity, and another end of the elastic portion abuts against the locking block.

5. The puncture locking device according to claim 4, wherein along the first direction, the first accommodating cavity has a first end surface closer to the suture needle and a second end surface farther from the suture needle, and the second end surface is inclined relative to the first direction, the inclined abutting wall is the second end surface.

6. The puncture locking device according to claim 4, wherein the elastic portion comprises a spring, the spring is arranged in the first accommodating cavity along an axial direction of the push-pull guiding member, and the spring is sleeved on the push-pull guide element.

7. The puncture locking device according to claim 4, wherein the elastic portion comprises a bent elastic sheet, the bent elastic sheet is arranged in the first accommodating cavity along a length direction of the push-pull guiding member, the bent elastic sheet comprises at least one first sub-plate, and the at least one first sub-plate is proximate to the locking block and elastically abuts against the locking block.

8. The puncture locking device according to claim 2, wherein in the unlocked state, the unlocking member extends through the suture head, and an end of the unlocking member is configured to push the locking block to separate the locking block from the push-pull guiding member.

9. The puncture locking device according to claim 1, wherein the elastic portion comprises an elastic main body arranged along a direction perpendicular to an axial direction of the push-pull guiding member, the locking portion is a locking hole arranged on the elastic main body, and the push-pull guiding member extends through the locking hole;
the suture head defines a second accommodating cavity configured for accommodating the elastic main body, and positions of an end of the elastic main body distal from the push-pull guiding member are limited on a wall of the second accommodating cavity;
the elastic main body comprises a first bent segment and a second bent segment, an angle is defined between the first bent segment and the second bent segment, and the locking hole is located at a junction of the first bent segment and the second bent segment; the unlocking member is configured to move on the junction of the first bent segment and the second bent segment, so that the junction is elastically deformed to adjust a size of the locking hole;
in the locked state, an inner wall of the locking hole abuts against the push-pull guiding member; in the unlocked state, the inner wall of the locking hole is separated from the push-pull guiding member.

10. The puncture locking device according to claim 2, wherein the unlocking member is a metal flexible tube structure, the unlocking member is sleeved over the push-pull guiding member, and the unlocking member and the push-pull guiding member are configured to independently move.

11. The puncture locking device according to claim 8, wherein the suture head defines a threaded hole, and the unlocking member is threadedly engaged within the threaded hole.

12. The puncture locking device according to claim 2, wherein the unlocking member is a pull wire, and the locking portion is configured to separate from the push-pull guiding member in response to the pull wire is pulled.

13. The puncture locking device according to claim 1, wherein the suture assembly further comprises a slider slidably arranged within the suture head, and the suture needle and the push-pull guiding member are respectively connected to the slider.

14. A suture apparatus, comprising:
a catheter;
a handheld operation portion arranged at a proximal end of the catheter; and
a puncture locking device comprising a suture head, a suture assembly and a locking structure arranged inside the suture head; wherein the suture assembly comprises a suture needle and a push-pull guiding member operatively coupled to the suture needle; the locking structure comprises an elastic locking assembly and an unlocking member, the elastic locking assembly comprises a locking portion configured to limit a position of the push-pull guiding member and an elastic portion configured to elastically deformed against the locking portion;
the suture head is arranged at a distal end of the catheter; the push-pull guiding member extends through the catheter and is configured to reciprocate along an axial direction of the catheter; the unlocking member is arranged through the catheter and is configured to move along the axial direction of the catheter; both of the push-pull guiding member and the unlocking member are connected to the handheld operation portion, and the handheld operation portion is configured to control a movement of the push-pull guiding member and the unlocking member;
when in a locked state, the elastic portion elastically deforms against the locking portion, the locking portion is tilted by the elastic portion and abuts against the push-pull guiding member, thereby restricting the movement of the push-pull guiding member along a first direction, and the first direction is a moving direction of the push-pull guiding member in response to the push-pull guiding member drives the suture needle to penetrate out of the suture head; and
when in an unlocked state, the unlocking member moves within the suture head to release the locking portion from tilting and abutting against the push-pull guiding member.

15. The suture apparatus according to claim 14, wherein an inclined abutting wall is arranged within the suture head, and the locking portion comprises a locking block; in the locked state, the elastic portion elastically deforms against the locking block to cause the locking block to abut against the inclined abutting wall, such that the locking block deflects is titled and abut against the push-pull guiding member.

16. The suture apparatus according to claim 15, wherein the locking block defines a via hole, the push-pull guiding member extends through the via hole, an outer wall of the push-pull guiding member and an inner wall of the via hole are engaged in a clearance fit manner; in the locked state, the locking block is titled to cause the push-pull guiding member to abut against the inner wall of the via hole of the locking block.

17. The suture apparatus according to claim 15, wherein the suture head defines a first accommodating cavity configured for accommodating the elastic locking assembly, and the inclined abutting wall is located within the first accommodating cavity; the push-pull guiding member extends through the first accommodating cavity; the elastic portion and the locking block are separated from each other, one end of the elastic portion abuts against an inner wall of the first accommodating cavity, and another end of the elastic portion abuts against the locking block.

18. The suture apparatus according to claim 17, wherein along the first direction, the first accommodating cavity has a first end surface closer to the suture needle and a second end surface farther from the suture needle, and the second end surface is inclined relative to the first direction, the inclined abutting wall is the second end surface.

19. The suture apparatus according to claim 17, wherein the elastic portion comprises a spring, the spring is arranged in the first accommodating cavity along an axial direction of the push-pull guiding member, and the spring is sleeved on the push-pull guide element.

20. The suture apparatus according to claim 17, wherein the elastic portion comprises a bent elastic sheet, the bent elastic sheet is arranged in the first accommodating cavity along a length direction of the push-pull guiding member, the bent elastic sheet comprises at least one first sub-plate, and the at least one first sub-plate is proximate to the locking block and elastically abuts against the locking block.
